(19) United States
(12) Patent Application Publication
(10) Pub. No.: US 2015/0093836 A1
(43) Pub. Date: Apr. 2, 2015

(54) METHOD FOR CONTROLLING DISSOCIATION OF DOUBLE STRANDED NUCLEIC ACID, METHOD FOR CONTROLLING STRAND EXCHANGE REACTION OF DOUBLE STRANDED NUCLEIC ACID AND METHOD FOR AMPLIFYING NUCLEIC ACID

(71) Applicants: SYSMEX CORPORATION, Kobe-shi (JP); NATIONAL UNIVERSITY CORPORATION NAGOYA UNIVERSITY, Nagoya-shi (JP)

(72) Inventors: Seigo SUZUKI, Kobe-shi (JP); Hiroya KIRIMURA, Kobe-shi (JP); Hiroyuki ASANUMA, Nagoya-shi (JP)

(73) Assignees: NATIONAL UNIVERSITY CORPORATION NAGOYA UNIVERSITY, Nagoya-shi (JP); SYSMEX CORPORATION, Kobe-shi (JP)

(21) Appl. No.: 14/497,603

(22) Filed: Sep. 26, 2014

(30) Foreign Application Priority Data
Sep. 30, 2013 (JP) 2013-204784

(51) Int. Cl. C12Q 1/68

(52) U.S. Cl. CPC C12Q 1/6811
USPC 436/94; 436/174

(57) ABSTRACT

The present invention relates to a method for controlling dissociation of a double stranded nucleic acid. The present invention also relates to a method for controlling strand exchange reaction of a double stranded nucleic acid. The present invention further relates to a method for amplifying a nucleic acid.

### CROSS-REFERENCE TO RELATED APPLICATION

This application claims priority to Japanese patent application No. 2013-204784 filed on Sep. 30, 2013, which is incorporated herein by reference in its entirety.

### BACKGROUND OF THE INVENTION

1. Technical Field

The present invention relates to a method for controlling dissociation of a double stranded nucleic acid. The present invention also relates to a method for controlling strand exchange reaction of a double stranded nucleic acid. The present invention further relates to a method for amplifying a nucleic acid.

2. Background

In various fields of research and medicine, nucleic acid amplification methods such as PCR (Polymerase chain reaction) and LAMP (Loop-mediated isothermal amplification) (see U.S. Pat. No. 6,410,278) and the like have been known in order to amplify target nucleic acids. In the nucleic acid amplification methods, a double stranded nucleic acid is dissociated into single strands to which primers are bound to be subjected to nucleic acid amplification.

In PCR, double stranded nucleic acids are dissociated by heating thereof to at or above 90° C. However, since the thermal reaction cycle of PCR is controlled in an automated manner, a thermal cycler is required in reality. In addition, heating to an increased temperature promotes deterioration of other components (such as enzymes) in reaction solutions.

In LAMP, the reaction proceeds under substantially isothermal conditions and a strand-displacing polymerase dissociates a double stranded nucleic acid between a strand extended from an inner primer and a template nucleic acid. However, LAMP requires inner primers having specific structures and reagents such as strand-displacing polymerases. In addition, because of the nature of the isothermal amplification, strand displacement reaction develops sequentially without being able to be controlled once the reaction is commenced.

### SUMMARY OF THE INVENTION

The scope of the present invention is defined solely by the appended claims, and is not affected to any degree by the statements within this summary.

The present invention provides a method for controlling dissociation of a double stranded nucleic acid, comprising steps of:

irradiating a photo-responsive nucleic acid with light having a first wavelength to make the photo-responsive nucleic acid capable of associating with a first nucleic acid that is one of strands in the double stranded nucleic acid,

wherein the double stranded nucleic acid comprises the first nucleic acid and a second nucleic acid having a sequence complementary to the first nucleic acid, and

wherein the photo-responsive nucleic acid has a sequence complementary to the first nucleic acid; and

allowing complementary association of the photo-responsive nucleic acid with the first nucleic acid to dissociate the second nucleic acid from the first nucleic acid.

Further, the present invention provides a method for controlling strand exchange reaction of a double stranded nucleic acid, comprising steps of:

irradiating a photo-responsive nucleic acid with light having a first wavelength to make the photo-responsive nucleic acid capable of associating with a first nucleic acid that is one of strands in the double stranded nucleic acid,

wherein the double stranded nucleic acid comprises the first nucleic acid and a second nucleic acid having a sequence complementary to the first nucleic acid, and

wherein the photo-responsive nucleic acid has a sequence complementary to the first nucleic acid;

allowing complementary association of the photo-responsive nucleic acid with the first nucleic acid to dissociate the second nucleic acid from the first nucleic acid;

irradiating the photo-responsive nucleic acid with light having a second wavelength that is different from the first wavelength to make the photo-responsive nucleic acid incapable of associating with the first nucleic acid;

dissociating the photo-responsive nucleic acid from the first nucleic acid; and

allowing complementary association of the first nucleic acid with a third nucleic acid.

Further, the present invention provides a method for amplifying nucleic acid by repeating following steps (1) to (4) under substantially isothermal condition, comprising steps of:

(1) allowing complementary association of a first polynucleotide with a single stranded target nucleic acid and allowing complementary association of a second polynucleotide with a region in the target nucleic acid that is adjacent to the region where the first polynucleotide associates;

(2) linking the first polynucleotide and the second polynucleotide;

(3) bringing a double stranded nucleic acid between the target nucleic acid and a linked strand of the first polynucleotide and the second polynucleotide into contact with a photo-responsive nucleic acid that has been made capable of associating with the target nucleic acid by irradiation with light having a first wavelength to allow complementary association of the target nucleic acid with the photo-responsive nucleic acid and to dissociate the linked strand; and

(4) irradiating the photo-responsive nucleic acid with light having a second wavelength that is different from the first wavelength to make the photo-responsive nucleic acid incapable of associating with the target nucleic acid and to dissociate the target nucleic acid from the photo-responsive nucleic acid,

wherein the dissociated target nucleic acid serves as the single stranded target nucleic acid in the step (1).

### BRIEF DESCRIPTION OF THE DRAWINGS

FIG. 1 is a conceptual diagram exemplifying a desirable reaction principle of an embodiment in relation to a method for controlling dissociation of a double stranded nucleic acid;

FIG. 2 is a conceptual diagram exemplifying photo-response of a photo-responsive nucleic acid containing azobenzene;

FIG. 3 is a conceptual diagram exemplifying a desirable reaction principle of another embodiment in relation to a method for controlling dissociation of a double stranded nucleic acid;

FIG. 4 is a conceptual diagram exemplifying a desirable reaction principle of an embodiment in relation to a method for controlling strand exchange reaction of a double stranded nucleic acid;

FIG. 5 is a conceptual diagram exemplifying a desirable reaction principle of another embodiment in relation to a method for controlling strand exchange reaction of a double stranded nucleic acid;

FIG. 6 is a conceptual diagram exemplifying a desirable reaction principle of an embodiment in relation to a method for amplifying a nucleic acid;

FIG. 7 is a conceptual diagram showing a reaction principle of Example 1 in relation to a method for controlling dissociation of a double stranded nucleic acid;

FIG. 8 is a graph showing that depending on irradiation with visible light and ultraviolet light, a double stranded nucleic acid is dissociated and re-associated;

FIG. 9A is a graph showing an effectiveness of a strand-exchange-enhancing substance in a method for controlling dissociation of a double stranded nucleic acid using a photo-responsive nucleic acid containing azobenzene;

FIG. 9B is a graph showing an effectiveness of a strand-exchange-enhancing substance in a method for controlling dissociation of a double stranded nucleic acid using a photo-responsive nucleic acid containing dimethylazobenzene;

FIG. 10A is a graph showing an effect of a low temperature condition in a method for controlling dissociation of a double stranded nucleic acid wherein a strand-exchange-enhancing substance is not used;

FIG. 10B is a graph showing an effect of a low temperature condition in a method for controlling dissociation of a double stranded nucleic acid wherein a strand-exchange-enhancing substance is used;

FIG. 11 is a graph showing an effectiveness of a high salt concentration condition in a method for controlling dissociation of a double stranded nucleic acid;

FIG. 12 is a graph showing sequence dependency of photo-responsive nucleic acids in a method for controlling dissociation of a double stranded nucleic acid;

FIG. 13 is a conceptual diagram showing a reaction principle of Example 6 in relation to a method for controlling strand exchange reaction of a double stranded nucleic acid;

FIG. 14 is a graph showing that strand exchange reaction of a double stranded nucleic acid occurs depending on irradiation with visible light and ultraviolet light;

FIG. 15A is a fluorescence image showing that amplification reaction of a nucleic acid occurs depending on the number of cycles of irradiation of visible light and ultraviolet light; and

FIG. 15B is a graph showing that amplification reaction of a nucleic acid occurs depending on the number of cycles of irradiation of visible light and ultraviolet light.

### DETAILED DESCRIPTION OF THE EMBODIMENTS

The preferred embodiments of the present invention will be described hereinafter with reference to the drawings.

In the method for controlling dissociation of a double stranded nucleic acid and the method for controlling strand exchange reaction of a double stranded nucleic acid according to the present invention, a photo-responsive nucleic acid is used and thus dissociation of the double stranded nucleic acid and progress of the strand exchange reaction can be controlled by irradiation with light. The control methods of the present invention do not require a heating step in order to denature and dissociate a double stranded nucleic acid or a cooling step in order to complementarily associate another nucleic acid strand with a dissociated strand of the double stranded nucleic acid. Thus the reaction system may be incubated substantially isothermally.

The reaction system means a defined field or space where factors required for a desired reaction exist and the reaction occurs. In the present invention, the reaction system may include, but is not limited to, for example a microdroplet such as a reaction solution or emulsion containing a double stranded nucleic acid and a photo-responsive nucleic acid and being accommodated in a container that allows transmission of light. The reaction system may contain an additive for providing an environment suitably allowing dissociation of a double stranded nucleic acid and strand exchange reaction. Such an additive is well known in the art and may include, for example, a buffer and a salt. The buffer is not particularly limited as far as it provides suitable pH for dissociation of a double stranded nucleic acid and strand exchange reaction and may include, for example, Tris-HCl, MES, phosphate buffers and the like. The salt may include, for example, NaCl, KCl, (NH₄)₂SO₄ and the like.

As used herein, the term “double stranded nucleic acid” refers to nucleic acids including a first nucleic acid which is a single stranded nucleic acid and a second nucleic acid associated therewith which is a single stranded nucleic acid complementary so as to be able to associate with the first nucleic acid. In the present specification, “double stranded nucleic acid” also encompasses nucleic acids having a stem-loop structure. In this case, one strand in the “stem” portion which is a double strand is designated as a first nucleic acid and the other strand is designated as a second nucleic acid.

As used herein, the expression “complementarily associate” or the like refers to the binding via a hydrogen bond of a whole or part of a polynucleotide to a whole or part of another polynucleotide under stringent conditions. In the present invention, “complementary association” and “hybridization” are synonymous in terms of formation of a double strand via a hydrogen bond. The “stringent condition” may be a condition commonly used by a person skilled in the art during hybridization of polynucleotides and may include the condition which allows specific hybridization of a polynucleotide to the other polynucleotide, wherein the polynucleotides have at least 90% and preferably at least 95% sequence identity therebetween. Stringency of hybridization is known to be a function of temperature, salt concentration, length and GC content of polynucleotides and concentration of a chaotropic agent in a hybridization buffer. The stringent condition may be the one described in, for example, Sambrook, J. et al., 1998, Molecular Cloning: A Laboratory Manual (2nd edition), Cold Spring Harbor Laboratory Press, New York.

As used herein, the term “base sequence fully complementary to” or the like refers to a base sequence of a polynucleotide that forms complementary base pairs of Watson-Click model with all bases in the other polynucleotide.

[Method for Controlling Dissociation of a Double Stranded Nucleic Acid]

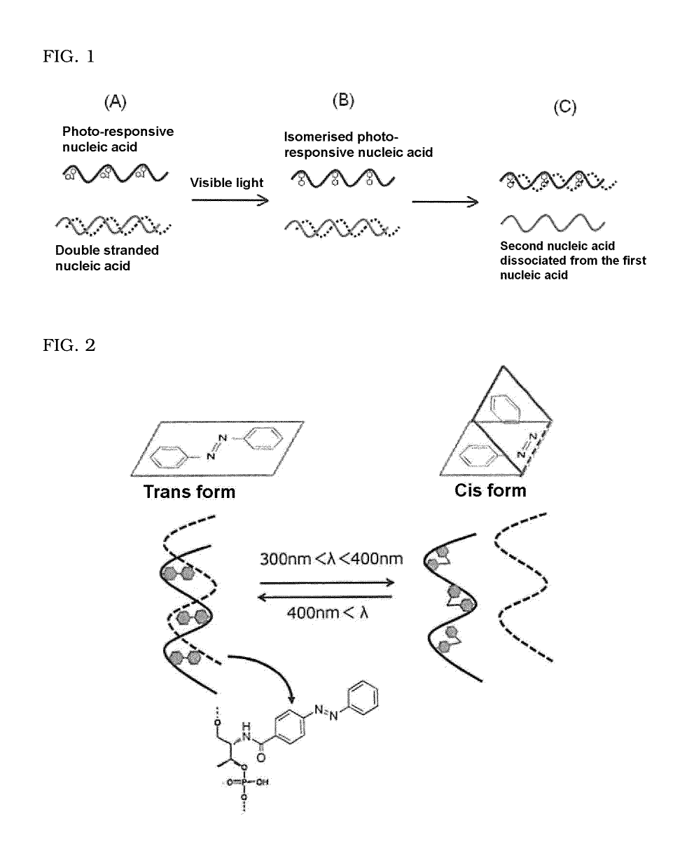

An embodiment (embodiment 1) of the method for controlling dissociation of a double stranded nucleic acid of the present invention, wherein two nucleic acid strands in a double stranded nucleic acid are dissociated by means of a photo-responsive nucleic acid is described hereinbelow. With regard to the method for controlling dissociation of a double stranded nucleic acid, among two nucleic acid strands in the double stranded nucleic acid, the strand which complementarily associates with a photo-responsive nucleic acid is for convenience sake designated as “first nucleic acid” and the strand which is dissociated from the first nucleic acid due to the association thereof with the photo-responsive nucleic acid is designated as “second nucleic acid”. See FIG. 1 which also exemplifies the desirable reaction principle of the embodiment 1. FIG. 1 shows the principle of the reaction wherein a photo-responsive nucleic acid containing azobenzene or a derivative thereof is used.

In the embodiment, a step of irradiating a photo-responsive nucleic acid with light having a first wavelength to make the photo-responsive nucleic acid capable of associating with a first nucleic acid of a double stranded nucleic acid is first carried out. By referring to FIG. 1, in this step, a photo-responsive nucleic acid which is in the form incapable of associating with the first nucleic acid (see (A) in FIG. 1) is converted to the form capable of associating with the first nucleic acid by irradiation with visible light (see (B) in FIG. 1).

In the present invention, the double stranded nucleic acid may be a double stranded DNA, a double stranded RNA or a hybrid of a single stranded RNA and a single stranded DNA. The double stranded nucleic acid may alternatively be a conventionally well known artificial nucleic acid such as PS-oligo, PNA (peptide nucleic acid), morpholino oligo, 2′O-substituted RNA and BNA (Bridged Nucleic Acid). The morphology of the double stranded nucleic acid is not particularly limited and may be a cyclic double stranded nucleic acid such as a plasmid DNA or a hairpin-like double stranded nucleic acid which is a single stranded nucleic acid having a self-complementary region in the molecule. The origin of the double stranded nucleic acid is not particularly limited and may be a naturally-occurring double stranded nucleic acid such as genomic DNA or a double stranded nucleic acid (e.g., a mRNA-cDNA hybrid and a double stranded cDNA) synthesized or amplified from a naturally-occurring nucleic acid. The double stranded nucleic acid may, as far as it serves as a template for synthesis of a complementary strand, be modified with a well known label substance or contain an artificial derivative of a nucleotide substituting a nucleotide in the nucleic acid.

As used herein, the term “photo-responsive nucleic acid” refers to a single stranded nucleic acid containing one or more organic groups which undergo isomerization and conformational change by irradiation with light having a certain wavelength. Nucleic acids containing such organic groups per se are well known in the art and may include, for example, photo-responsive oligonucleotides described in WO 01/21637. In the present invention, complementary association with one of strands in a double stranded nucleic acid and dissociation from the associated strand can be reversibly carried out by utilizing the conformational change of the organic group in the photo-responsive nucleic acid by irradiation with light.

The nucleic acid used for the photo-responsive nucleic acid may be DNA or RNA, or may be a conventionally well known artificial nucleic acid such as PS-oligo, PNA (peptide nucleic acid), morpholino oligo, 2′O-substituted RNA and BNA (Bridged Nucleic Acid), among which DNA is preferred.

The manner of binding of a nucleic acid to an organic group which may confer photo responsiveness to the nucleic acid (hereinafter also referred to as “photo-responsive organic group”) in the photo-responsive nucleic acid is not particularly limited as far as the organic group binds to the nucleic acid so that the organic group forms a side chain of the nucleic acid. A side chain of a nucleic acid is a moiety corresponding to a base diverging from a pentose in a nucleotide constituting the nucleic acid. A backbone of a nucleic acid is a strand of nucleotides constituting the nucleic acid containing linkages between pentoses and phosphates. In the present invention, a photo-responsive organic group is regarded as being attached as a side moiety of a nucleic acid when the photo-responsive organic group binds to a 5′-terminal nucleotide or a 3′-terminal nucleotide of the nucleic acid. Examples of the manner of binding of a photo-responsive organic group to a nucleic acid may include direct binding of the organic group to a nucleotide so that the organic group is a side moiety of the nucleic acid or indirect binding of the organic group to the nucleic acid through an appropriate intervening group inserted into the backbone of the nucleic acid. The intervening group may be appropriately selected by a person skilled in the art and may include, for example, an alkylene group having 1 to 10, preferably 1 to 6 carbon atoms or a group containing an amino acid or a derivative thereof.

The photo-responsive organic group is suitably a group which can reversibly isomerise, by irradiation with light having a certain wavelength, from a substantially planar structure to a nonplanar structure. A compound that can be used as the organic group may include, for example, azobenzene, stilbene, spiropyran and derivatives thereof. It has been known that azobenzene and stilbene isomerise from trans forms to cis forms by irradiation with light and that spiropyran isomerises from a merocyanine form to a spiropyran form by irradiation with light.

In the present invention, the photo-responsive nucleic acid is preferably a nucleic acid containing a photo-responsive organic group that increases the melting temperature (Tm) of the nucleic acid compared to the Tm of a nucleic acid with an identical base sequence. Such a photo-responsive nucleic acid is suitably a nucleic acid containing one or more groups of at least one species selected from azobenzene and a derivative thereof. The derivative of azobenzene is not particularly limited as far as it does not prevent formation of double strands and is particularly preferably dimethylazobenzene because it hardly undergoes isomerisation by heat. A nucleic acid containing azobenzene or a derivative thereof per se is well known in the art and can be commonly produced or is generally available.

Azobenzene or a derivative thereof takes a planar trans form by irradiation with visible light having a wavelength of 400 nm or more and takes a three-dimensional cis form by irradiation with ultraviolet light having a wavelength of 300 to 400 nm. Thus by irradiation with visible light, azobenzene or a derivative thereof in the photo-responsive nucleic acid takes a planar trans form that does not prevent formation of double strands and thus the photo-responsive nucleic acid complementarily associates with a given nucleic acid strand to form a double strand. Meanwhile by irradiation with ultraviolet light, azobenzene or a derivative thereof in the photo-responsive nucleic acid takes a three-dimensional cis form, resulting in steric hindrance that prevents formation of a double strand. Due to this the photo-responsive nucleic acid is dissociated from the given nucleic acid in a double strand. See FIG. 2 which shows a model for formation and dissociation of a double strand using a photo-responsive nucleic acid containing azobenzene or a derivative thereof. The photo-responsive nucleic acid shown in FIG. 2 contains azobenzene which is attached via D-threoninol inserted in the backbone of the nucleic acid so as to form a side chain moiety of the nucleic acid, which however does not limit the present invention.

Thus when a photo-responsive nucleic acid containing azobenzene or a derivative thereof is used in the method of the present invention, the photo-responsive nucleic acid can take the form that is capable of associating with the first nucleic acid by irradiation with light having a first wavelength that is visible light having a wavelength of 400 nm or more.

The number of a photo-responsive organic group in a photo-responsive nucleic acid is not particularly limited as far as a double strand can be dissociated due to isomerisation. For example, one photo-responsive organic group may be introduced at every 2 to 10 bases in a photo-responsive nucleic acid.

When referring to a base sequence of a photo-responsive nucleic acid herein, a photo-responsive organic group attached to the nucleic acid may be disregarded and base portions of nucleotides are only looked at as a base sequence of general nucleic acids. For example, with regard to a photo-responsive nucleic acid containing a plurality of groups of azobenzene or a derivative thereof attached to a given single stranded nucleic acid, the base sequence of the photo-responsive nucleic acid is considered to be the same as that of the given single stranded nucleic acid.

The base sequence of the photo-responsive nucleic acid is not particularly limited as far as it allows complementary association with the first nucleic acid. However, the photo-responsive nucleic acid preferably has a base sequence that is fully complementary to the base sequence of the first nucleic acid.

The photo-responsive nucleic acid may have any length as far as the nucleic acid can maintain complementary association with the first nucleic acid and is usually 10 to 100 nucleotides in length and preferably 15 to 50 nucleotides in length. In the method for controlling strand exchange reaction of a double stranded nucleic acid as described hereinbelow, a region in the double stranded nucleic acid with which a third nucleic acid described hereinbelow can associate varies depending on the base sequence and length of the photo-responsive nucleic acid. Thus it is preferable that the photo-responsive nucleic acid is designed according to a region for which strand exchange is sought in the double stranded nucleic acid.

The photo-responsive nucleic acid may be optionally labelled with a well known label substance. The nucleic acid may be labelled with a radioisotope or a non-radioactive substance. The radioisotope may include ³²P, ³³P, ³⁵S, ³H and ¹²⁵I. The non-radioactive substance may include ligands such as biotin, avidin, streptavidin or digoxigenin, haptens, pigments, quenchers and luminescent reagents such as chemiluminescent, bioluminescent, fluorescent or phosphorescent reagents.

In the present invention, the wavelength (first wavelength and second wavelength) of light irradiated may be appropriately selected according to the type of the photo-responsive organic group. The period of irradiation of light may also be appropriately selected according to the type of the photo-responsive organic group and may be, in case of azobenzene or a derivative thereof, generally 5 to 300 seconds and preferably 10 to 60 seconds. The light source is not particularly limited as far as it can irradiate the reaction system with light having a predetermined wavelength and may include, for example, in case of azobenzene or a derivative thereof, a combination of a mercury lamp and a visible light filter or a LED having a predetermined wavelength.

In the present embodiment, a step of allowing complementary association of the photo-responsive nucleic acid which is capable of associating with the nucleic acid strand as described above with the first nucleic acid to dissociate the second nucleic acid in the double stranded nucleic acid is carried out. By referring to FIG. 1, strand exchange between the double stranded nucleic acid and the photo-responsive nucleic acid (see (B) in FIG. 1) allows formation of a double strand between the photo-responsive nucleic acid and the first nucleic acid and dissociation of the second nucleic acid from the first nucleic acid (see (C) in FIG. 1).

This step is desirably carried out under the situation that allows strand exchange of the second nucleic acid by the photo-responsive nucleic acid. This situation can be created under the condition under which both formation of hydrogen bonds and dissociation occur between base pairs in the double stranded nucleic acid. The condition may vary according to the length of the double stranded nucleic acid and may include, but is not limited to, incubation of the reaction system at 45 to 70° C. and preferably 55 to 60° C. in case of 40 bases or addition of a strand-exchange-enhancing substance described hereinafter to the reaction system. Under this condition, two nucleic acid strands in the double stranded nucleic acid dissociate and the photo-responsive nucleic acid can complementarily associated with the first nucleic acid of the double stranded nucleic acid due to the complementarity of the base sequences. Accordingly the second nucleic acid of the double stranded nucleic acid can be dissociated.

The length of the portion in the second nucleic acid that dissociates from the first nucleic acid depends on the length of the photo-responsive nucleic acid (more specifically, the length of the portion in the photo-responsive nucleic acid which complementarily associates with the first nucleic acid). Namely according to the present invention, when the photo-responsive nucleic acid has a length that is shorter than the double stranded nucleic acid, a part of the second nucleic acid is dissociated. When the photo-responsive nucleic acid has a length that is the same as or longer than the double stranded nucleic acid, the whole second nucleic acid is dissociated.

In the present invention, in order to allow effective association of the photo-responsive nucleic acid with the first nucleic acid, it is preferable that the concentration of the photo-responsive nucleic acid is higher than the concentration of the double stranded nucleic acid. Specific concentrations of the nucleic acids may be appropriately selected by a person skilled in the art and it is particularly preferable that the concentration of the photo-responsive nucleic acid is 10 times or more higher than the concentration of the double stranded nucleic acid.

In order to allow effective association of the photo-responsive nucleic acid with the first nucleic acid, it is also advantageous to carry out the method for controlling dissociation of a double stranded nucleic acid under a high salt concentration condition. The salt is not particularly limited as far as it does not damage the nucleic acids and may include, for example, NaCl, KCl and the like. The salt concentration is desirably within the range that can maintain complementary association between two nucleic acid strands and may be, for example, a concentration up to 2 M when NaCl is used.

Further, in order to allow effective association of the photo-responsive nucleic acid with the first nucleic acid, it is also advantageous to carry out the method for controlling dissociation of a double stranded nucleic acid in the presence of a well known strand-exchange-enhancing substance. The strand-exchange-enhancing substance is well known in the art and may include, for example, at least one selected from a cationic homopolymer and a cationic copolymer. The cationic homopolymer and cationic copolymer may include, for example, amino acids such as lysine, arginine and histidine, saccharides such as glucosamine, homopolymers and copolymers derived from monomers that can form a cationic group such as synthetic monomers including ethyleneimine, diethylaminoethyl methacrylate, dimethylaminoethyl methacrylate and the like.

The cationic homopolymer or copolymer preferably has a graft structure containing a hydrophilic polymer modifying on a side chain. The side chain (grafted chain) is formed by, for example, at least one water-soluble polymer selected from the group consisting of water-soluble polyalkylene glycols such as polyethylene glycol; water-soluble polysaccharides such as dextran, pullulan, amylose and arabinogalactan; water-soluble polyamino acids containing hydrophilic amino acids such as serine, asparagine, glutamine and threonine; water-soluble polymers synthesized with monomers of acrylamide and a derivative thereof; water-soluble polymers synthesized with monomers of methacrylic acid, acrylic acid and a derivative thereof (e.g., hydroxyethyl methacrylate); polyvinyl alcohols and derivatives thereof. The molecular weight of the cationic homopolymer or copolymer and the chain length and degree of grafting of the side chain modification group are not particularly limited and may be appropriately selected by a person skilled in the art.

The strand-exchange-enhancing substance is particularly preferably a poly(L-lysine)-graft-dextran copolymer (PLL-g-Dex) which is a cationic copolymer. PLL-g-Dex per se is disclosed in Japanese Unexamined Patent Application Publication No. 2001-78769, which is incorporated herein by reference in its entirety.

The concentration of the strand-exchange-enhancing substance in the reaction system is not particularly limited as far as dissociation of the nucleic acid or strand exchange reaction is not inhibited and can be appropriately selected by a person skilled in the art.

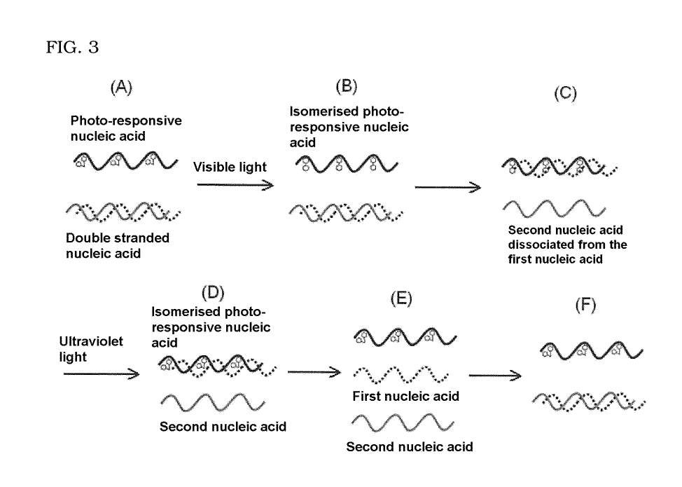

In the embodiment 1, the second nucleic acid in the double stranded nucleic acid is dissociated while the first nucleic acid in the double stranded nucleic acid and the photo-responsive nucleic acid are remained to be associated. With regard to the method for controlling dissociation of a double stranded nucleic acid, an embodiment (embodiment 2) including a step of dissociating the photo-responsive nucleic acid from the first nucleic acid is thus described hereinafter. See FIG. 3 which exemplifies the desirable reaction principle of the embodiment 2. FIG. 3 shows the principle of the reaction wherein a photo-responsive nucleic acid containing azobenzene is used.

In the embodiment, a step of irradiating a photo-responsive nucleic acid with light having a first wavelength to make the photo-responsive nucleic acid capable of associating with a first nucleic acid is first carried out (see (A) and (B) in FIG. 3). A step of allowing complementary association of the photo-responsive nucleic acid with the first nucleic acid to dissociate the second nucleic acid in the double stranded nucleic acid is then carried out (see (C) in FIG. 3). The details for the steps are the same as those described above for the embodiment 1.

In the embodiment, a step of irradiating the photo-responsive nucleic acid with light having a second wavelength to make the photo-responsive nucleic acid incapable of associating with the first nucleic acid is carried out (see (D) in FIG. 3).

The light having a second wavelength may be light that has a different wavelength from the light having a first wavelength used in the step of making the photo-responsive nucleic acid capable of associating with the first nucleic acid and that can sterically isomerise an organic group in the photo-responsive nucleic acid, so that the photo-responsive nucleic acid cannot associate with the nucleic acid strand. When the photo-responsive nucleic acid used contains azobenzene or a derivative thereof for example, irradiation with light having a second wavelength that is ultraviolet light having a wavelength of 300 to 400 nm can transform azobenzene to have a cis form, resulting in the photo-responsive nucleic acid incapable of associating with the first nucleic acid.

The interval between irradiation with light having a first wavelength and irradiation of light having a second wavelength may be appropriately selected without particular limitation and may be usually 1 to 300 seconds and preferably 5 to 60 seconds. The period of irradiation of light having a second wavelength may be appropriately selected according to the type of the organic group in the photo-responsive nucleic acid, and may be, in case of azobenzene or a derivative thereof, generally 1 to 60 seconds and preferably 5 to 30 seconds. The light source is not particularly limited as far as it can irradiate the reaction system with light having a predetermined wavelength and may include, for example, in case of azobenzene or a derivative thereof, a combination of a mercury lamp and an ultraviolet light filter or a LED having a predetermined wavelength.

In the embodiment, a step of allowing dissociation of the first nucleic acid from the photo-responsive nucleic acid made incapable of associating with the first nucleic acid is carried out. In this step, due to steric hindrance of the organic group in the photo-responsive nucleic acid, a hydrogen bond between a base in the photo-responsive nucleic acid and a base in the first nucleic acid of the double stranded nucleic acid cannot be maintained, resulting in dissociation of the photo-responsive nucleic acid from the first nucleic acid (see (E) in FIG. 3). Accordingly in the embodiment a whole or part of the double stranded nucleic acid dissociates depending on the length of the photo-responsive nucleic acid and the photo-responsive nucleic acid is released.

The released photo-responsive nucleic acid is now incapable of associating with nucleic acid strands, so that two nucleic acid strands (the first nucleic acid and the second nucleic acid) can re-associate to restore the original double stranded nucleic acid (see (F) in FIG. 3). Thus in case of repeating the dissociation reaction of the embodiment, light having a first wavelength may be irradiated generally 1 to 300 seconds and preferably 5 to 60 seconds after irradiation of light having a second wavelength.

[Method for Controlling Strand Exchange Reaction of a Double Stranded Nucleic Acid]

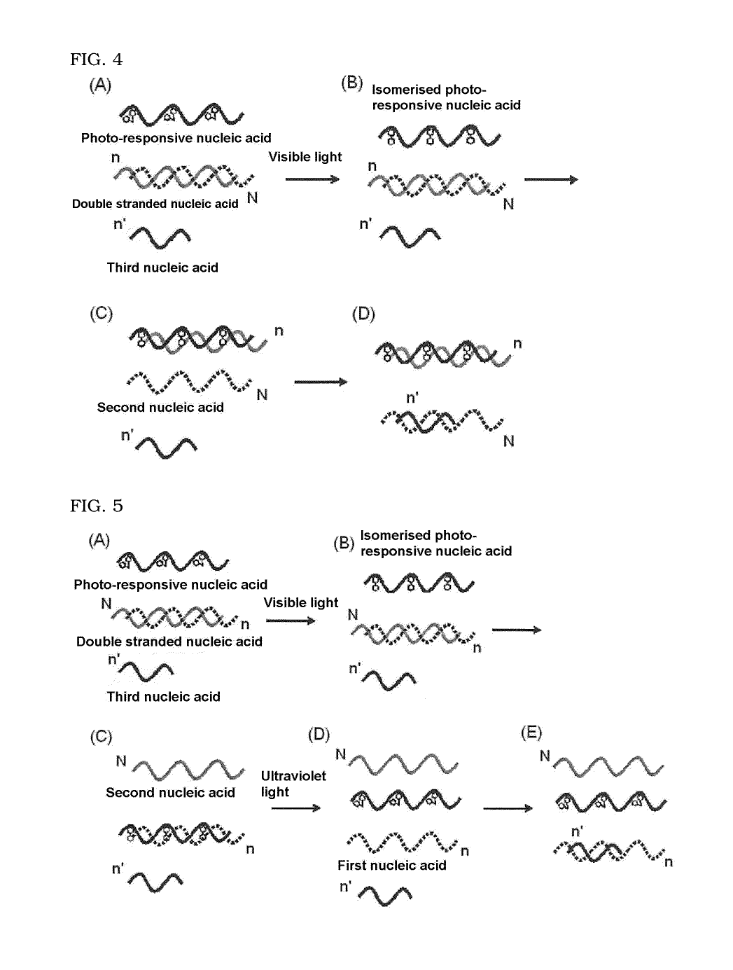

In the present invention, strand exchange reaction of a double stranded nucleic acid can also be controlled by utilizing the method for controlling dissociation of a double stranded nucleic acid with the photo-responsive nucleic acid and light irradiation. Namely the scope of the invention encompasses a method for controlling strand exchange reaction of a double stranded nucleic acid. With regard to the method for controlling strand exchange reaction of a double stranded nucleic acid, among two nucleic acid strands constituting the double stranded nucleic acid, the strand which complementarily associates with a photo-responsive nucleic acid is for convenience sake designated as “first nucleic acid” and the strand which is dissociated from the first nucleic acid due to the association is designated as “second nucleic acid”. The nucleic acid to replace the first nucleic acid or the second nucleic acid is designated as “third nucleic acid”. An embodiment (embodiment 1) wherein the second nucleic acid and the third nucleic acid are complementarily associated by using the photo-responsive nucleic acid is hereinafter described. See FIG. 4 which exemplifies the desirable reaction principle of the embodiment 1. FIG. 4 shows the principle of the reaction wherein a photo-responsive nucleic acid containing azobenzene or a derivative thereof is used.

In the embodiment, a step of irradiating the photo-responsive nucleic acid with light having a first wavelength to make the photo-responsive nucleic acid capable of associating with a first nucleic acid of a double stranded nucleic acid is carried out. By referring to FIG. 4, in this step, a photo-responsive nucleic acid which is in the form incapable of associating with the first nucleic acid is (see (A) in FIG. 4) converted to be capable of associating with the first nucleic acid by irradiation with visible light (see (B) in FIG. 4).

In the embodiment, among two nucleic acid strands in the double stranded nucleic acid, it is the first nucleic acid that is replaced by the third nucleic acid. The region which is replaced in the first nucleic acid may account for a portion or a whole of the first nucleic acid, depending on the length of the photo-responsive nucleic acid (more specifically, the length of the portion in the photo-responsive nucleic acid which complementarily associates with the first nucleic acid). Namely according to the present invention, when the photo-responsive nucleic acid has a length that is shorter than the double stranded nucleic acid, the region to be replaced is a part of the first nucleic acid. To the contrary, when the photo-responsive nucleic acid has a length that is the same as or longer than the double stranded nucleic acid, the whole first nucleic acid is replaced. In FIG. 4, the first nucleic acid and the second nucleic acid in the double stranded nucleic acid are respectively designated as “n” and “N” and the third nucleic acid as “n”.

In the embodiment, the base sequence of the photo-responsive nucleic acid is not particularly limited as far as it allows complementary association with the first nucleic acid. However, the photo-responsive nucleic acid preferably has a base sequence that is fully complementary to the base sequence of the first nucleic acid. The details for the photo-responsive nucleic acid and light to be irradiated are the same as those described above for the method for controlling dissociation of a double stranded nucleic acid.

In the embodiment, a step of allowing complementary association of the photo-responsive nucleic acid which is capable of associating with the first nucleic acid as described above with the first nucleic acid in the double stranded nucleic acid to dissociate the second nucleic acid in the double stranded nucleic acid is carried out (see (B) and (C) in FIG. 4).

In the embodiment, the step of dissociating the first nucleic acid from the complementary strand thereof, i.e., the second nucleic acid by means of the photo-responsive nucleic acid is substantially the same as the dissociation step in the method for controlling dissociation of a double stranded nucleic acid as described above. Accordingly the details for the reaction conditions and additives (e.g., salts and the strand-exchange-enhancing substance) in this step are the same as those described above for the method for controlling dissociation of a double stranded nucleic acid.

As shown in (C) in FIG. 4, after this step, the reaction system contains the photo-responsive nucleic acid associated with the first nucleic acid of the double stranded nucleic acid and the released second nucleic acid. The third nucleic acid is also released in the reaction system.

In the embodiment, a step of allowing complementary association of the second nucleic acid with the third nucleic acid is carried out. As described above, in this step, the second nucleic acid among two nucleic acid strands in the double stranded nucleic acid complementarily associates with the third nucleic acid by strand exchange reaction (see (D) in FIG. 4).

In the embodiment, the third nucleic acid is not particularly limited as far as it is an oligonucleotide that can complementarily associate with the second nucleic acid under stringent conditions and is preferably an oligonucleotide having a base sequence fully complementary to the second nucleic acid. The length of the third nucleic acid can be appropriately selected depending on the length of the second nucleic acid. The third nucleic acid can be produced according to a synthesis method of nucleic acids well known in the art.

In the embodiment, the photo-responsive nucleic acid has the base sequence that can complementarily associate with the first nucleic acid of the double stranded nucleic acid and the third nucleic acid has the base sequence that can complementarily associate with the second nucleic acid. Accordingly the third nucleic acid may complementarily associate with the photo-responsive nucleic acid. Thus in order to reduce the possibility of complementary association of the third nucleic acid with the photo-responsive nucleic acid, the third nucleic acid may be designed, for example, such that the length of the portion in the third nucleic acid which complementarily associates with the second nucleic acid is shorter than the length of the photo-responsive nucleic acid.

In the embodiment, one or more species of the third nucleic acid may be used. When two or more species of the third nucleic acid are used, it is desirable that they are designed so as to associate with different regions in the second nucleic acid. The regions in the second nucleic acid with which two or more species of the third nucleic acid associate may be adjacent or apart from each other.

In the present invention, the third nucleic acid may be labelled with a well known label substance. The details for the label for the third nucleic acid are the same as those described for the label for the photo-responsive nucleic acid. The third nucleic acid may contain an oligonucleotide of a functional sequence such as a recognition sequence by a certain restriction enzyme or a tag sequence attached to the 5′-terminal and/or 3′-terminal thereof.

An embodiment (embodiment 2) wherein the first nucleic acid is allowed to complementarily associate with the third nucleic acid by means of the photo-responsive nucleic acid is now described hereinafter. See FIG. 5 which exemplifies the desirable reaction principle of the embodiment 2. FIG. 5 shows the principle of the reaction wherein a photo-responsive nucleic acid containing azobenzene or a derivative thereof is used.

In the embodiment, a step of irradiating a photo-responsive nucleic acid with light having a first wavelength to make the photo-responsive nucleic acid capable of associating with a first nucleic acid of a double stranded nucleic acid is first carried out. By referring to FIG. 5, in this step, a photo-responsive nucleic acid which is in the form incapable of associating with the first nucleic acid (see (A) in FIG. 5) is converted to the form capable of associating with the first nucleic acid by irradiation with visible light (see (B) in FIG. 5).

In the embodiment, among two nucleic acid strands in the double stranded nucleic acid, it is the second nucleic acid that is replaced by the third nucleic acid. The region which is replaced in the second nucleic acid may account for a portion or a whole of the second nucleic acid, depending on the length of the photo-responsive nucleic acid (more specifically, the length of the portion in the photo-responsive nucleic acid which complementarily associates with the first nucleic acid). Namely according to the present invention, when the photo-responsive nucleic acid has a length that is shorter than the double stranded nucleic acid, the region to be replaced is a part of the second nucleic acid. To the contrary, when the photo-responsive nucleic acid has a length that is the same as or longer than the double stranded nucleic acid, the whole second nucleic acid is replaced. In FIG. 5, the first nucleic acid and the second nucleic acid in the double stranded nucleic acid are respectively designated as “n” and “N” and the third nucleic acid as “n”.

The details for the photo-responsive nucleic acid used in the embodiment are the same as those described above for the embodiment 1. The details for the photo-responsive nucleic acid and light to be irradiated are the same as those described above for the method for controlling dissociation of a double stranded nucleic acid.

In the embodiment, a step of allowing complementary association of the photo-responsive nucleic acid which is capable of associating with the first nucleic acid as described above with the first nucleic acid to dissociate the second nucleic acid in the double stranded nucleic acid is carried out (see (B) and (C) in FIG. 5).

In the embodiment, the step of dissociating the first nucleic acid from the complementary strand thereof, i.e., the second nucleic acid by means of the photo-responsive nucleic acid is the same as the embodiment 1. Accordingly the details for the reaction conditions and additives (e.g., salts and the strand-exchange-enhancing substance) in this step are the same as those described above for the method for controlling dissociation of a double stranded nucleic acid.

In the embodiment, a step of irradiating the photo-responsive nucleic acid with light having a second wavelength to make the photo-responsive nucleic acid incapable of associating with the first nucleic acid is carried out ((C) and (D) in FIG. 5).

This step is the same as the method for controlling dissociation of a double stranded nucleic acid described above in that the organic group in the photo-responsive nucleic acid is sterically isomerised by irradiation with light having a second wavelength to make the photo-responsive nucleic acid incapable of associating with the first nucleic acid. Accordingly, the details for light having a first wavelength and light having a second wavelength are the same as those described above for the method for controlling dissociation of a double stranded nucleic acid.

In the embodiment, a step of allowing dissociation of the first nucleic acid from the photo-responsive nucleic acid made incapable of associating with the first nucleic acid as described above is carried out (see (D) in FIG. 5).

In this step, due to steric hindrance of the organic group in the photo-responsive nucleic acid, a hydrogen bond between a base in the photo-responsive nucleic acid and a base in the first nucleic acid of the double stranded nucleic acid cannot be maintained, resulting in dissociation of the photo-responsive nucleic acid from the first nucleic acid. Accordingly after this step, the reaction system contains the double stranded nucleic acid a part or whole of which is dissociated, and the released photo-responsive nucleic acid and the third nucleic acid (see (D) in FIG. 5).

In the embodiment, a step of allowing complementary association of the first nucleic acid dissociated as described above with the third nucleic acid is carried out (see (E) in FIG. 5). In the embodiment, the third nucleic acid is not particularly limited as far as it is an oligonucleotide that can be complementarily associated with the first nucleic acid under stringent conditions and is preferably an oligonucleotide having a base sequence fully complementary to the first nucleic acid. The length of the third nucleic acid can be appropriately selected depending on the length of the first nucleic acid. The third nucleic acid can be produced according to a synthesis method of nucleic acids well known in the art.

The photo-responsive nucleic acid released in the previous step is now incapable of associating with the nucleic acid strands, so that the two nucleic acid strands (first nucleic acid and second nucleic acid) can re-associate to restore the original double stranded nucleic acid. However, the restoration of the original double stranded nucleic acid does not allow strand exchange reaction. In order to promote association with the third nucleic acid in the embodiment, it is desirable that the concentration of the third nucleic acid at the initiation of strand exchange reaction is higher than the concentration of the double stranded nucleic acid. Specific concentrations of the nucleic acids may be appropriately selected by a person skilled in the art and it is particularly preferable that the concentration of the third nucleic acid is 10 times or more higher than the concentration of the double stranded nucleic acid.

[Method for Amplifying a Nucleic Acid]

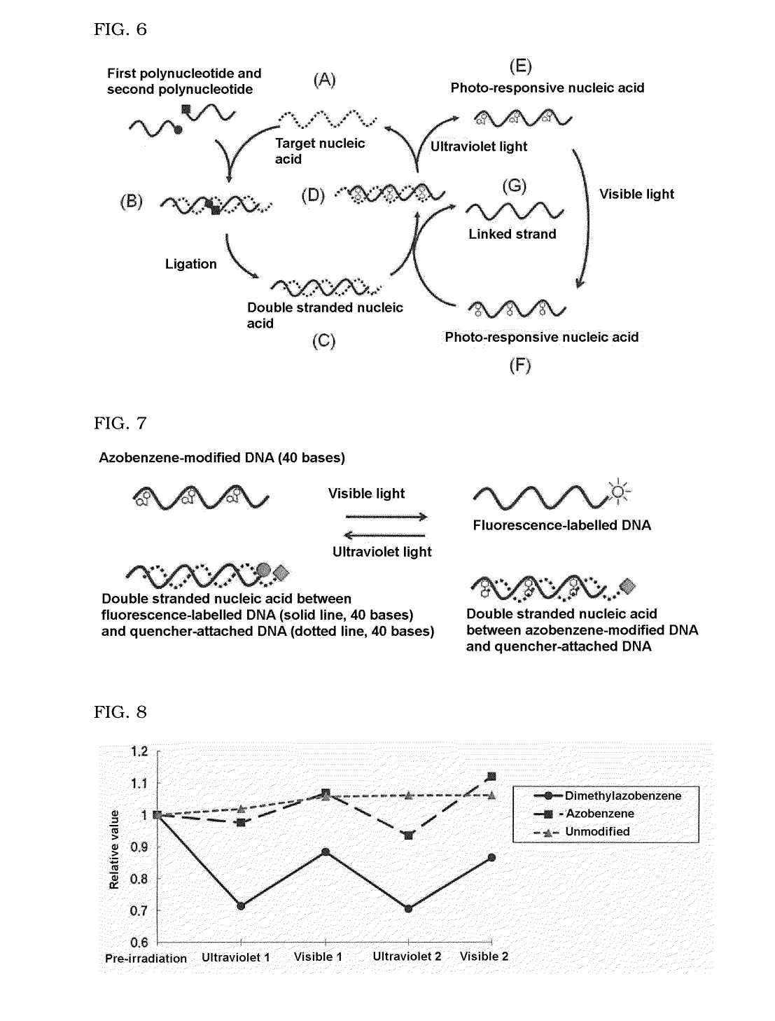

In the present invention, nucleic acids can also be amplified by utilizing the methods for controlling dissociation of a double stranded nucleic acid and controlling strand exchange reaction with the photo-responsive nucleic acid and light irradiation. Specifically, dissociation of a double stranded nucleic acid and strand exchange reaction of the present invention can be used for a method for amplifying a nucleic acid in which a plurality of linkable nucleic acid probes for a target nucleic acid that is one strand among two nucleic acid strands of the double stranded nucleic acid is used to form a linked nucleic acid. The method for amplifying a nucleic acid of the present invention (hereinafter also referred to as “amplification method”) is thus hereinafter described. In the amplification method of the present invention, a nucleic acid can be amplified whether the template during sample preparation is a double stranded nucleic acid or a single stranded nucleic acid. However, for explanation sake, nucleic acid amplification reaction in which the template during sample preparation is a single stranded nucleic acid (hereinafter also referred to as “single stranded target nucleic acid”) is described hereinafter. See FIG. 6 which exemplifies the desired reaction principle of the amplification method. FIG. 6 shows the principle of the reaction wherein a photo-responsive nucleic acid containing azobenzene or a derivative thereof is used.

In the amplification method of the present invention, a step of allowing binding of a first polynucleotide to a single stranded target nucleic acid and allowing binding of a second polynucleotide to a region in the target nucleic acid that is adjacent to the region to which the first polynucleotide binds is carried out (see (A) and (B) in FIG. 6). In this step, when the first polynucleotide and the second polynucleotide complementarily associate with the target nucleic acid, the 3′-terminal of one of the polynucleotides is adjacent to the 5′-terminal of the other polynucleotide so that the terminals may be linked.

In the amplification method of the present invention, the first polynucleotide and the second polynucleotide are polynucleotides which hybridize with the single stranded target nucleic acid under stringent conditions and contain bases at the 5′-terminal and 3′-terminal that form base pairs with bases in the target nucleic acid. The first polynucleotide and the second polynucleotide may be designed so that they are adjacent according to the base sequence and the length of the region for which amplification is sought. The polynucleotides may be respectively labelled with a well known label substance as far as the following linking step is not inhibited. The details for the label substance are the same as those described above for the photo-responsive nucleic acid.

In the amplification method of the present invention, a step of linking the first polynucleotide and the second polynucleotide is carried out (see (B) and (C) in FIG. 6). The obtained linked strand of the first polynucleotide and the second polynucleotide may be hereinafter merely referred to as “linked strand”. The means for linking the polynucleotides is not particularly limited and may be selected from well known means in the art. The probes may be linked by, for example, enzyme reactions, chemical ligation reactions, photo-ligation reactions and the like.

When two adjacent polynucleotides are linked by enzyme reaction, a ligase which is well-known in the art and generally used for ligation of nucleic acids may be used. The ligase may include, for example, T4 DNA ligase, Tth DNA ligase and the like.

When two adjacent polynucleotides are linked by chemical ligation reaction, well known organic groups which bind each other by chemical reaction may be attached to the 5′-terminal of one polynucleotide and the 3′-terminal of the other polynucleotide. The organic groups may include, for example, coupling reagents such as N-cyanoimidazole and cyanogen bromide, a combination of a phosphorothioate group and a iodo group and the like. Chemical ligation reaction per se is well known in the art and is described, for example, in US Patent Publication No. 2008/124810.

When two adjacent polynucleotides are linked by photo-ligation reaction, well known organic groups which bind each other by irradiation with light may be attached to the 5′-terminal of one polynucleotide and the 3′-terminal of the other polynucleotide. The organic groups may include, for example, a carboxyvinyl group. Specifically, a nucleic acid having a pyrimidine base at the 5′-terminal that contains a carboxyvinyl group at the 5-position can bind to a nucleic acid having cytosine at the 3′-terminal by irradiation with light. Photo-ligation reaction per se is well known in the art and described, for example, in Japanese Unexamined Patent Application Publication No. 2001-348398.

In the amplification method of the present invention, a step of bringing a double stranded nucleic acid of the target nucleic acid and the linked strand into contact with a photo-responsive nucleic acid that has been made capable of associating with the target nucleic acid by irradiation with light having a first wavelength (see (E) and (F) in FIG. 6) to allow complementary association of the target nucleic acid with the photo-responsive nucleic acid (see (D) in FIG. 6) and to dissociate the linked strand (see (G) in FIG. 6).

The base sequence of the photo-responsive nucleic acid is not particularly limited as far as it allows complementary association with the target nucleic acid. However, the photo-responsive nucleic acid preferably has a base sequence that is fully complementary to the base sequence of the target nucleic acid. The details for the photo-responsive nucleic acid and light to be irradiated are the same as those described above for the method for controlling dissociation of a double stranded nucleic acid. The details for strand exchange reaction with the photo-responsive nucleic acid are the same as those described above for the method for controlling dissociation of a double stranded nucleic acid and the method for controlling strand exchange reaction.

In the amplification method of the present invention, a step of irradiating the photo-responsive nucleic acid with light having a second wavelength that is different from the first wavelength to make the photo-responsive nucleic acid incapable of associating with the target nucleic acid and to dissociate the target nucleic acid from the photo-responsive nucleic acid (see (D), (E) and (A) in FIG. 6), resulting in the dissociated target nucleic acid being the single stranded target nucleic acid in the first step (see (A) and (B) in FIG. 6). The details for light to be irradiated and dissociation of nucleic acids using the photo-responsive nucleic acid are the same as those described above for the method for controlling dissociation of a double stranded nucleic acid.

When the amplification reaction is repeated in the amplification method of the present invention, light having a first wavelength may be irradiated generally 1 second to 30 minutes and preferably 5 to 60 seconds after irradiation with light having a second wavelength.

In the amplification method of the present invention, an amplification product may be detected by electrophoresis and the like which are conventionally known. The amplification product may alternatively be detected based on a signal generated from the label substance attached to the first polynucleotide and/or the second polynucleotide. When the label substance is a fluorescent substance or a radioisotope, fluorescence or radiation may be measured after electrophoresis of the amplification product. The fluorescent substance may be indirectly attached to the polynucleotide. For example, attachment of the fluorescent substance through avidin and biotin, attachment of the fluorescent substance through hapten and an anti-hapten antibody and the like may be mentioned. Specifically, in case of attachment of the fluorescent substance through avidin and biotin, a probe attached to biotin and a fluorescent substance attached to avidin or streptavidin may be brought into contact to label the probe with the fluorescent substance.

Among a plurality of probes, the first polynucleotide and the second polynucleotide may be respectively labelled with fluorescent substances that undergo fluorescence resonance energy transfer (FRET). For example, one fluorescent substance may be provided so as to quench the other fluorescent substance when the first polynucleotide and the second polynucleotide are linked and the fluorescent substances are in proximity. The fluorescent substances may also be provided so that FRET occurs when the fluorescent substances are in proximity and the fluorescent substances are excited at a wavelength that is different from a wavelength for excitation of the fluorescent substances which are not in proximity.

In the method for controlling dissociation of a double stranded nucleic acid, the method for controlling strand exchange reaction of a double stranded nucleic acid and the method for amplifying a nucleic acid, two or more species of the photo-responsive nucleic acid may be used. For example, when two species of the photo-responsive nucleic acids are used, a second photo-responsive nucleic acid can further be used that associates with the region in the first nucleic acid of the double stranded nucleic acid that is different from the region with which a first photo-responsive nucleic acid associates. The region with which the first photo-responsive nucleic acid associates and the region with which the second photo-responsive nucleic acid associates may be distant or adjacent in the base sequence of the first nucleic acid in the double stranded nucleic acid. Use of two or more species of the photo-responsive nucleic acids allows accurate dissociation of the double stranded nucleic acid even though the double stranded nucleic acid has a long sequence.

The present invention is hereinafter described more specifically by way of Examples which do not limit the present invention.

### EXAMPLES

### Example 1

### Control of Dissociation of Double Stranded Nucleic Acid Using Photo-Responsive Nucleic Acid

In the present Example, the photo-responsive nucleic acid was used to evaluate whether or not a double stranded nucleic acid could be dissociated by irradiation with light. The reaction principle of the present Example is exemplified in FIG. 7. FIG. 7 shows the principle of the reaction wherein a photo-responsive nucleic acid containing azobenzene is used.

(1) Preparation of Measurement Sample

### (1-1) Photo-Responsive Nucleic Acid

A single stranded DNA containing one azobenzene or 2′,6′-dimethylazobenzene attached at every 2 bases was synthesized as a photo-responsive nucleic acid by Tsukuba Oligo Service Co., Ltd. The sequence of the photo-responsive nucleic acid is shown below:

(SEQ ID NOs: 1 and 2)
5′-CT(Z)TT(Z)AA(Z)GA(Z)AG(Z)GA(Z)GA(Z)TA(Z)TA(Z)
CC(Z)TG(Z)AG(Z)TG(Z)AT(Z)CT(Z)AG(Z)TG(Z)TA(Z)CT
(Z)TA-3′

In the above sequence, (Z) represents a site where azobenzene or 2′,6′-dimethylazobenzene is inserted. Azobenzene and 2′,6′-dimethylazobenzene are attached so as to form side chain moieties of the nucleic acid through D-threoninol integrated into the backbone of the single stranded DNA.

As a negative control for the photo-responsive nucleic acid, an unmodified single stranded DNA with the same base sequence of 40 bases as the photo-responsive nucleic acid was synthesized by Life Technologies Japan. The sequence of the single stranded DNA is shown below:

(SEQ ID NO: 3)
5′-CTTTAAGAAGGAGATATACCTGAGTGATCTAGTGTACTTA-3′

### (1-2) Double Stranded Nucleic Acid

A double stranded nucleic acid was prepared by annealing a single stranded DNA labelled with a fluorescent substance and a single stranded DNA labelled with a quencher. In the double stranded nucleic acid, a signal from the fluorescent substance is quenched due to the presence of the quencher when two nucleic acid strands are not dissociated, while a signal is released from the fluorescent substance when two nucleic acid strands are dissociated. Thus whether the double stranded nucleic acid is dissociated or not can be evaluated by measurement of the fluorescence intensity of a sample.

A single stranded DNA labelled with FITC at the 5′-terminal was synthesized as a single stranded DNA labelled with a fluorescent substance by Japan Bio Services Co., Ltd. A single stranded DNA labelled with Black Hole Quencher™ 1 (BHQ1) at the 3′-terminal was synthesized as a single stranded DNA labelled with a quencher by Japan Bio Services Co., Ltd. The sequences of the single stranded DNAs are shown below:

(SEQ ID NO: 4)
5′-FITC-TTTAAGAAGGAGATATACCTGAGTGATCTAGTGTAC
TTA-3′
(SEQ ID NO: 5)
5′-TTAAGTACACTAGATCACTCAGGTATATCTCCTTCTTAAAG-
BHQ1-3′

The double stranded nucleic acid was prepared as follows. The FITC-labelled DNA and the BHQ 1-labelled DNA were dissolved at final concentrations of 90 nM and 100 nM, respectively, in 10 mM phosphate buffer (pH 7) containing sodium chloride (0.15 M) and dithiothreitol (1 mM). The obtained solution was heated to 85° C. and then cooled to 4° C. under the cooling condition of 1° C./min to obtain a solution of the double stranded nucleic acid of the FITC-labelled DNA and the BHQ1-labelled DNA.

### (1-3) Measurement Sample

The obtained solution of the double stranded nucleic acid was distributed to tubes. To the tubes were added the photo-responsive nucleic acids irradiated with ultraviolet light (azobenzene-modified DNA and dimethylazobenzene-modified DNA) and the unmodified single stranded DNA at a final concentration of 120 nM, respectively. Mineral oil was dropped to the tubes to give measurement samples.

(2) Dissociation of Double Stranded Nucleic Acid

The tubes containing measurement samples were heated to 59° C. in a stainless-steel tube rack on a Thermo Plate (Tokai Hit Corporation). The tubes were mounted on a stage of a fluorescence microscope (BX51, Olympus Corporation) and irradiated 30 seconds with light (wavelength: 470 to 495 nm) which was emitted from a light source of a mercury lamp (ultra-high pressure UV lamp USH-1030L, Olympus Corporation) and passed through a visible light filter (U-MNIBA3, Olympus Corporation). As a result, fluorescent images originated from FITC were obtained (referred to as “pre-irradiation”). At five minutes after the measurements, the tubes were irradiated 30 seconds with light (wavelength: 330 to 385 nm) which was emitted from the mercury lamp and passed through an ultraviolet light filter (U-MWU2, Olympus Corporation). After five minutes, fluorescent images originated from FITC were obtained (referred to as “ultraviolet 1”). At five minutes after the measurements, the tubes were irradiated 30 seconds with light (wavelength: 470 to 495 nm) which was emitted from the mercury lamp and passed through the visible light filter. After five minutes, fluorescent images originated from FITC were obtained (referred to as “visible 1”). At five minutes after the measurements, the tubes were irradiated 30 seconds with light (wavelength: 330 to 385 nm) which was emitted from the mercury lamp and passed through the ultraviolet light filter. After five minutes, fluorescent images originated from FITC were obtained (referred to as “ultraviolet 2”). At five minutes after the measurements, the tubes were irradiated 30 seconds with light (wavelength: 470 to 495 nm) which was emitted from the mercury lamp and passed through the visible light filter. After five minutes, fluorescent images originated from FITC were obtained (referred to as “visible 2”).

The resulting fluorescent images were converted to tif files and the intensity of fluorescent signals was converted to the numerical values with the Image J software (available from the website of National Institute of Health (NIH)). The relative values of the fluorescence intensity after irradiation with light were calculated for the measurement samples with the fluorescence intensity of the “pre-irradiation” being 1. The results are shown in FIG. 8. The melting temperature of the photo-responsive nucleic acid and the negative control unmodified DNA during irradiation with visible light is shown in Table 1.

TABLE 1

| | Tm (° C.) |
|---|---|
| Dimethylazobenzene-modified DNA | 83.7 |
| Azobenzene-modified DNA | 72.9 |
| Unmodified DNA | 65.8 |

(3) Results

In FIG. 8, the sample added with the unmodified DNA showed almost no change in the fluorescence intensity after irradiation with light having any wavelength. On the other hand, the samples added with the azobenzene- and dimethylazobenzene-modified photo-responsive nucleic acids showed a decrease and an increase in the fluorescence intensity by irradiation with ultraviolet light and visible light, respectively. An increase in the fluorescence intensity by irradiation with visible light means that the photo-responsive nucleic acid allowed dissociation of the FITC-labelled DNA strand from the double stranded nucleic acid by irradiation with visible light, resulting in generation of a fluorescent signal originated from FITC. A decrease in the fluorescence intensity by irradiation with ultraviolet light means that the photo-responsive nucleic acid could not maintain association thereof with the BHQ 1-labelled DNA strand in the double stranded nucleic acid by irradiation with ultraviolet light, resulting in re-formation of the original double stranded nucleic acid and elimination of the fluorescent signal originated from FITC. Thus it was found that the photo-responsive nucleic acid containing azobenzene or dimethylazobenzene could reversibly control dissociation of the double stranded nucleic acid in a light irradiation-dependent manner.

It was apparent that efficiency of dissociation was the highest when the photo-responsive nucleic acid containing dimethylazobenzene was used. In this context, the dimethylazobenzene-modified DNA had the highest Tm among the photo-responsive nucleic acids. Therefore it was suggested that use of the photo-responsive nucleic acid having high Tm may be effective for dissociation of double stranded nucleic acids by irradiation with light.

### Example 2

### Evaluation of Effectiveness of Strand-Exchange-Enhancing Substance in Dissociation of Double Stranded Nucleic Acid

In the present Example, whether or not use of a strand-exchange-enhancing substance is effective in dissociation of a double stranded nucleic acid by irradiation with light using a photo-responsive nucleic acid was evaluated.

(1) Preparation of Measurement Sample

In the present Example, the photo-responsive nucleic acids, the unmodified DNA and the double stranded nucleic acid which were the same as those in Example 1 were used. The measurement sample was prepared in the same manner as Example 1 except that a strand-exchange-enhancing substance, PLL-g-Dex (molecular weight of PLL: 8000, graft ratio: 90%), was added at a final concentration of 15.6 μM. For comparison, a measurement sample without addition of PLL-g-Dex was also prepared.

(2) Dissociation of Double Stranded Nucleic Acid

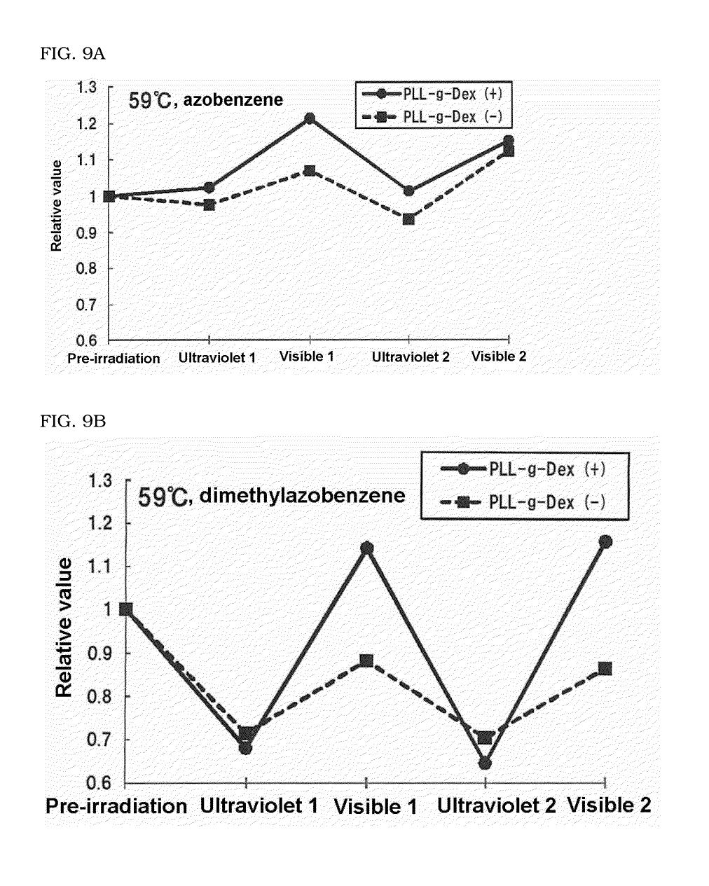

Fluorescent images originated from FITC for the prepared measurement samples were obtained by irradiating with light having the respective wavelengths in the same manner as Example 1. The resulting fluorescent images were converted to tif files and the intensity of fluorescent signals was converted to the numerical values with the Image J software. The relative values of the fluorescence intensity after irradiation with light were calculated for the measurement samples with the fluorescence intensity of the “pre-irradiation” being 1. The results are shown in FIGS. 9A and 9B.

(3) Results

In the presence of PLL-g-Dex, the change in the fluorescence intensity by irradiation with light was significantly increased compared to the absence of PLL-g-Dex. Specifically, the fluorescence intensity was significantly increased immediately after irradiation with visible light and the fluorescence intensity was then significantly decreased by irradiation with ultraviolet light. The fluorescence intensity was then again significantly increased by irradiation with visible light. Therefore it was apparent that in the presence of PLL-g-Dex, dissociation and re-formation of the double strand was more significantly induced, so that dissociation of the double stranded nucleic acid could be reversibly controlled by irradiation with light. As Example 1, it was also apparent that the efficiency of dissociation was highest when the photo-responsive nucleic acid containing dimethylazobenzene was used.

### Example 3

### Evaluation of Effects of Temperature Conditions on Dissociation of Double Stranded Nucleic Acid

In the present Example, whether or not dissociation of the double stranded nucleic acid could be controlled by irradiation with light under a condition of temperature (45° C.) that was lower than Examples 1 and 2 was evaluated.

(1) Preparation of Measurement Samples

In the present Example, the photo-responsive nucleic acids, the unmodified DNA and the double stranded nucleic acid which were the same as those in Example 1 were used. The measurement sample was prepared in the same manner as Example 1 except that a strand-exchange-enhancing substance, PLL-g-Dex (molecular weight of PLL: 8000, graft ratio: 90%), was added at a final concentration of 15.6 μM. For comparison, a measurement sample without addition of PLL-g-Dex was also prepared.

(2) Dissociation of Double Stranded Nucleic Acid

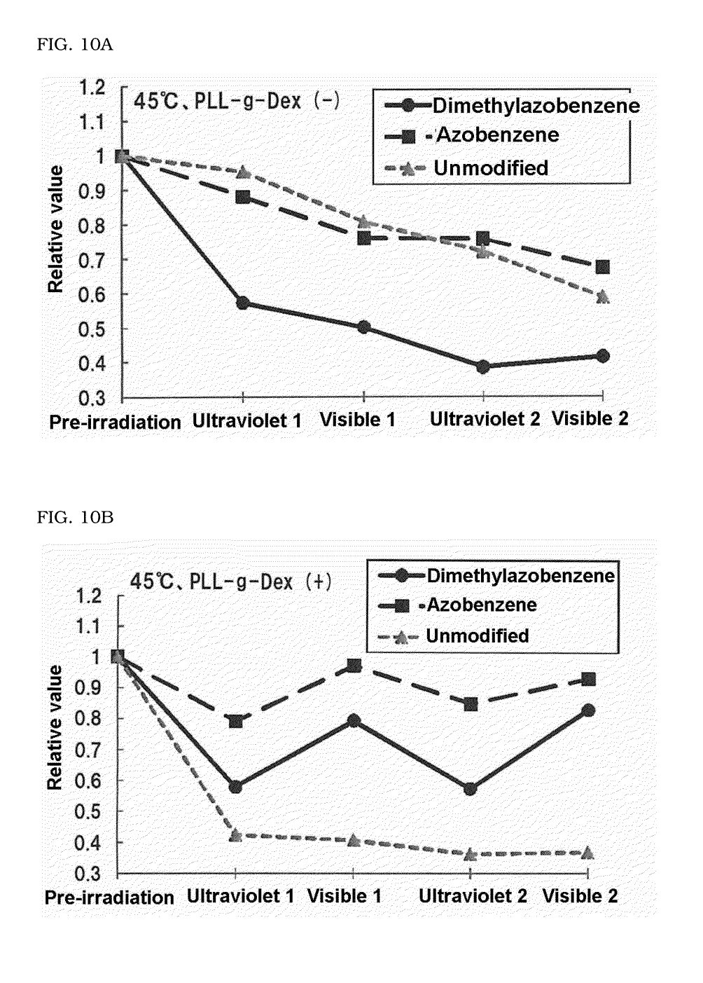

Fluorescent images originated from FITC for the prepared measurement samples were obtained by irradiating with light having the respective wavelengths in the same manner as Example 1 except that the tubes containing the measurement samples were heated to 45° C. The resulting fluorescent images were converted to tif files and the intensity of fluorescent signals was converted to the numerical values with the Image J software. The relative values of the fluorescence intensity after irradiation with light were calculated for the measurement samples with the fluorescence intensity of the “pre-irradiation” being 1. The results are shown in FIGS. 10A and 10B.

(3) Results

In the absence of the strand-exchange-enhancing substance or PLL-g-Dex, no change in the fluorescence intensity according to the cycles of irradiation with ultraviolet light and visible light was observed. On the other hand, in the presence of PLL-g-Dex, the change in the fluorescence intensity was observed which was dependent on light irradiation. Thus it was suggested that used of PLL-g-Dex may allow reversible control of dissociation of the double stranded nucleic acid by irradiation with light even under a low temperature condition.

### Example 4

### Evaluation of Effectiveness of High Salt Concentration on Dissociation of Double Stranded Nucleic Acid

In the present Example, whether a high salt concentration condition was useful or not was evaluated on dissociation of a double stranded nucleic acid by irradiation with light using a photo-responsive nucleic acid.

(1) Preparation of Measurement Samples

### (1-1) Photo-Responsive Nucleic Acid and Double Stranded Nucleic Acid

In the present Example, the photo-responsive nucleic acid (dimethylazobenzene-modified DNA) and the double stranded nucleic acid which were the same as those in Example 1 were used, except that in order to prepare measurement samples having high salt concentration, the double stranded nucleic acid used was obtained by dissolving the FITC-labelled DNA and the BHQ 1-labelled DNA of Example 1 in 10 mM phosphate buffer (pH 7) containing sodium chloride (1 M) and dithiothreitol (1 mM) at final concentrations of 90 nM and 100 nM, respectively.

### (1-2) Preparation of Measurement Samples

Three samples were prepared as follows. All the samples contained the dimethylazobenzene-modified DNA at a final concentration of 120 nM.

A sample containing PLL-g-Dex (molecular weight of PLL: 8000, graft ratio: 90%, final concentration: 15.6 μM) at a normal salt concentration (0.15 M sodium chloride);

A sample without PLL-g-Dex at a high salt concentration (1 M sodium chloride); and

A sample without PLL-g-Dex at a normal salt concentration (0.15 M sodium chloride).

(2) Dissociation of Double Stranded Nucleic Acid

The tubes containing measurement samples were heated to 59° C. in a stainless-steel tube rack on a Thermo Plate (Tokai Hit Corporation). The tubes were mounted on a stage of a fluorescence microscope (BX51, Olympus Corporation) and irradiated 30 seconds with light (wavelength: 470 to 495 nm) which was emitted from a light source of a mercury lamp (ultra-high pressure UV lamp USH-1030L, Olympus Corporation) and passed through a visible light filter (U-MNIBA3, Olympus Corporation). As a result, fluorescent images originated from FITC were obtained (referred to as “pre-irradiation”). The tubes were then irradiated 30 seconds with light (wavelength: 470 to 495 nm) which was emitted from a light source of the mercury lamp and passed through the visible light filter. After five minutes, fluorescent images originated from FITC were obtained (referred to as “post-visible irradiation”). At five minutes after the measurements, the tubes were irradiated 30 seconds with light (wavelength: 330 to 385 nm) which was emitted from a light source of the mercury lamp and passed through an ultraviolet light filter (U-MWU2, Olympus Corporation). After five minutes, fluorescent images originated from FITC were obtained (referred to as “post-UV irradiation”).

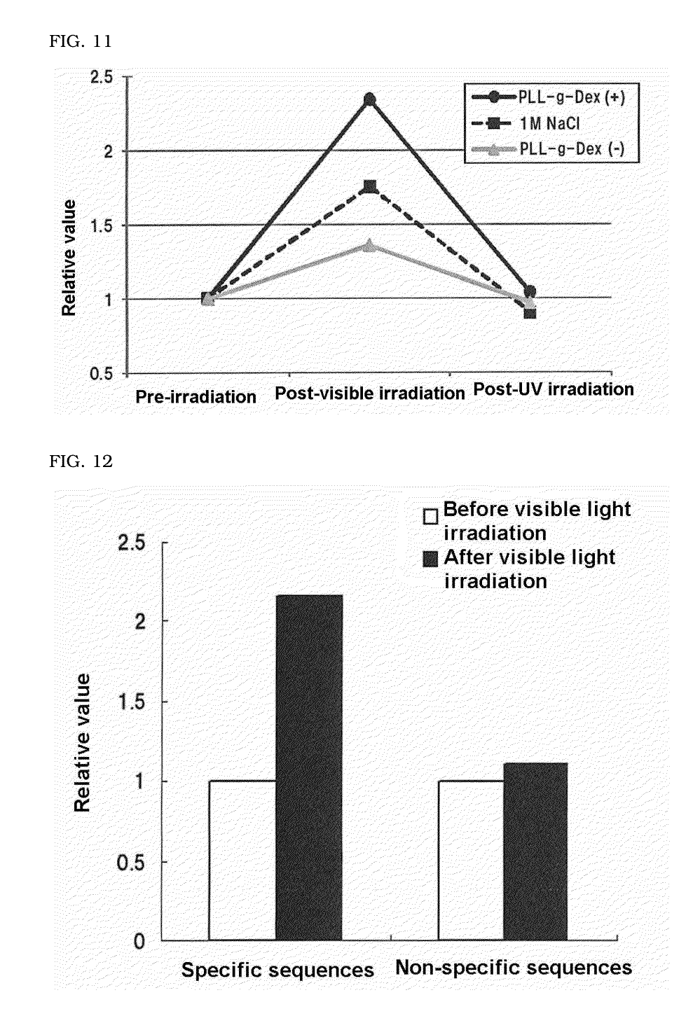

The resulting fluorescent images were converted to tif files and the intensity of fluorescent signals was converted to the numerical values with the Image J software. The relative values of the fluorescence intensity after irradiation with light were calculated for the measurement samples with the fluorescence intensity of the “pre-irradiation” being 1. The results are shown in FIG. 11.

(3) Results

Compared to the change in the fluorescence intensity by irradiation with light in the absence of PLL-g-Dex, the change in the fluorescence intensity by irradiation with light in the presence of 1 M sodium chloride was significantly increased. This suggests that dissociation of the double stranded nucleic acid by irradiation with light can also be controlled by increasing the salt concentration of the reaction system instead of using the strand-exchange-enhancing substance.

### Example 5

### Evaluation of Sequence Specificity of Photo-Responsive Nucleic Acid

In the present Example, whether or not the specificity of the photo-responsive nucleic acid towards the base sequence of a double stranded nucleic acid is important on dissociation of the double stranded nucleic acid was evaluated.

(1) Preparation of Measurement Samples

### (1-1) Photo-Responsive Nucleic Acid and Double Stranded Nucleic Acid

In the present Example, the photo-responsive nucleic acid (dimethylazobenzene-modified DNA) and the double stranded nucleic acid which were the same as those in Example 1 were used. The double stranded nucleic acid of Example 1 is also referred to as “double stranded nucleic acid having specific sequences” for convenience sake. In order to prepare a double stranded nucleic acid (hereinafter referred to as “double stranded nucleic acid having non-specific sequences”) having base sequences that are not complementary to the base sequence of the photo-responsive nucleic acid, labelled single stranded DNAs having the following base sequences were synthesized by Japan Bio Services, Co., Ltd.

(SEQ ID NO: 6)
5′-FITC-CAGATTACGATTCAGGTAAGGCTTAGACTTGAAAACCG
GT-3′
(SEQ ID NO: 7)
5′-ACCGGTTTTCAAGTCTAAGCCTTACCTGAATCGTAATCTG-
BHQ1-3′

The double stranded nucleic acid having non-specific sequences was prepared as follows. The FITC-labelled DNA and the BHQ1-labelled DNA were dissolved at final concentrations of 90 nM and 100 nM, respectively, in 10 mM phosphate buffer (pH 7) containing sodium chloride (0.15 M) and dithiothreitol (1 mM). The obtained solution was heated to 85° C. and then cooled to 4° C. under the cooling condition of 1° C./min to obtain a solution of the double stranded nucleic acid having non-specific sequences.

### (1-2) Preparation of Measurement Samples

The measurement samples which were a sample containing the double stranded nucleic acid having specific sequences and a sample containing the double stranded nucleic acid having non-specific sequences were prepared in the same manner as Example 1. All the samples contained the dimethylazobenzene-modified DNA (final concentration: 120 nM) and PLL-g-Dex (molecular weight of PLL: 8000, graft ratio: 90%, final concentration: 15.6 μM).

(2) Dissociation of Double Stranded Nucleic Acid

Fluorescent images originated from FITC for the prepared measurement samples were obtained by irradiating with light having the respective wavelengths in the same manner as Example 4. The resulting fluorescent images were converted to tif files and the intensity of fluorescent signals was converted to the numerical values with the Image J software. The relative values of the fluorescence intensity after irradiation with light were calculated for the measurement samples with the fluorescence intensity of the “pre-irradiation” being 1. The results are shown in FIG. 12.

(3) Results

The double stranded nucleic acid having specific sequences showed an increase in the fluorescence intensity after irradiation with visible light, similar to the results from the previous Examples. On the other hand, the double stranded nucleic acid having non-specific sequences did not at all show a change in the fluorescence intensity after irradiation with visible light. This revealed that dissociation of the double stranded nucleic acid using the photo-responsive nucleic acid is dependent on the base sequence of the double stranded nucleic acid rather than independent of the base sequence.

### Example 6

### Control of Strand Exchange Reaction Using Photo-Responsive Nucleic Acid

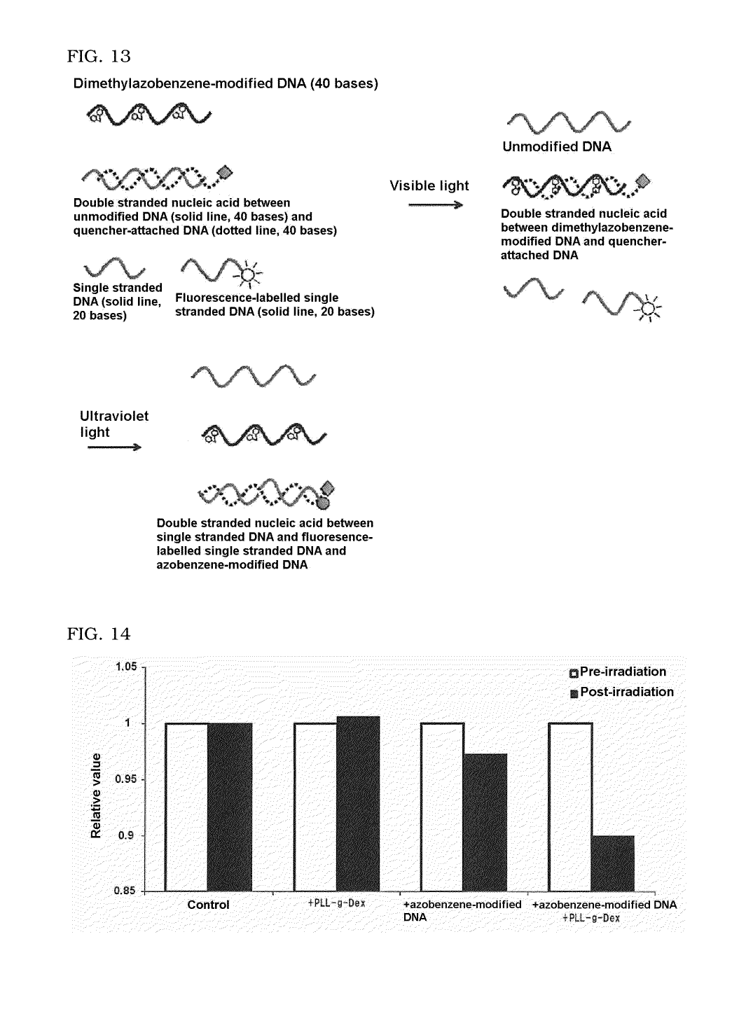

In the present Example, whether or not the strand exchange reaction of a double stranded nucleic acid by irradiation with light could be controlled by using a photo-responsive nucleic acid was evaluated. The reaction principle of the present Example is shown in FIG. 13.

(1) Preparation of Measurement Samples

### (1-1) Photo-Responsive Nucleic Acid

In the present Example, the photo-responsive nucleic acid (dimethylazobenzene-modified DNA) as Example 1 was used.

### (1-2) Double Stranded Nucleic Acid

A double stranded nucleic acid was prepared by annealing the unmodified single stranded DNA with the same base sequence of 40 bases as the photo-responsive nucleic acid and the single stranded DNA labelled with a quencher. The unmodified single stranded DNA and the single stranded DNA labelled with a Black Hole Quencher™ 2 (BHQ2) at the 5′-terminal were synthesized by Japan Bio Services Co., Ltd. The sequences of the single stranded DNAs are shown below:

(SEQ ID NO: 3)
5′-CTTTAAGAAGGAGATATACCTGAGTGATCTAGTGTACTTA-3′
(SEQ ID NO: 8)
5′-BHQ2-TTAAGTACACTAGATCACTCAGGTATATCTCCTTCTTAA
AG-3′

The double stranded nucleic acid was prepared as follows. The unmodified DNA and the BHQ2-labelled DNA were dissolved at final concentrations of 90 nM and 100 nM, respectively, in 10 mM phosphate buffer (pH 7) containing sodium chloride (0.15 M) and dithiothreitol (1 mM). The obtained solution was heated to 85° C. and then cooled to 4° C. under the cooling condition of 1° C./min to obtain a solution of the double stranded nucleic acid of the unmodified DNA and the BHQ2-labelled DNA.

### (1-3) Single Stranded Replacing Nucleic Acid (Third Nucleic Acid)

As single stranded replacing nucleic acids, an unmodified single stranded DNA having 20 bases and a single stranded DNA having 20 bases labelled with TexasRed® at the 3′-terminal were used. The single stranded DNAs have the same base sequence as SEQ ID NO: 3 as described above when the single stranded DNAs are linked. The labelled single stranded DNA was synthesized by Life Technologies Japan. The unmodified single stranded DNA having 20 bases was synthesized by Japan Bio Services, Co., Ltd. The sequences of the single stranded DNAs are shown below:

(SEQ ID NO: 9)
5′-CTTTAAGAAGGAGATATACC-3′
(SEQ ID NO: 10)
5′-TGAGTGATCTAGTGTACTTA-TexasRed®-3′

In the present Example, when the unmodified DNA strand in the double stranded nucleic acid is replaced by the single stranded replacing nucleic acid labelled with TexasRed®, the signal originated from TexasRed® is eliminated due to BHQ2 in the double stranded nucleic acid. Thus whether the strand exchange reaction has occurred or not can be evaluated by measuring the fluorescence intensity of a sample.

### (1-4) Preparation of Measurement Samples

The obtained solution of the double stranded nucleic acid was distributed to tubes. To the tubes were added the single stranded replacing nucleic acid (final concentration: 100 nM). One of the tubes served as a control sample without the photo-responsive nucleic acid or PLL-g-Dex. To the rest of the tubes were appropriately added the photo-responsive nucleic acid and/or PLL-g-Dex (molecular weight of PLL: 8000, graft ratio: 90%) to prepare the following three samples.

A sample containing PLL-g-Dex (final concentration: 15.6 μM) without the photo-responsive nucleic acid;

A sample containing the photo-responsive nucleic acid (final concentration: 120 nM) without PLL-g-Dex; and

A sample containing the photo-responsive nucleic acid (final concentration: 120 nM) and PLL-g-Dex (final concentration: 15.6 μM).

Mineral oil was dropped to the tubes to give measurement samples.

(2) Dissociation of Double Stranded Nucleic Acid

The tubes containing the measurement samples were heated to 60° C. in a stainless-steel tube rack on a Thermo Plate (Tokai Hit Corporation). The tubes were mounted on a stage of a fluorescence microscope (BX51, Olympus Corporation) and irradiated 30 seconds with light (wavelength: 470 to 495 nm) which was emitted from a light source of a mercury lamp (ultra-high pressure UV lamp USH-1030L, Olympus Corporation) and passed through a visible light filter (U-MNIBA3, Olympus Corporation). As a result, fluorescent images originated from TexasRed® were obtained (referred to as “pre-irradiation”). The tubes were then irradiated 30 seconds with light (wavelength: 470 to 495 nm) which was emitted from a light source of the mercury lamp and passed through the visible light filter. At 10 minutes after irradiation with visible light, the tubes were irradiated 30 seconds with light (wavelength: 330 to 385 nm) which was emitted from a light source of the mercury lamp and passed through the ultraviolet light filter. After 10 minutes, fluorescent image originated from TexasRed® were obtained (referred to as “post-irradiation”).

The resulting fluorescent images were converted to tif files and the intensity of fluorescent signals was converted to the numerical values with the Image J software. The relative values of the fluorescence intensity after irradiation with light were calculated for the measurement samples with the fluorescence intensity of the “pre-irradiation” being 1. The results are shown in FIG. 14.

(3) Results

The control sample and the sample containing only PLL-g-Dex did not show a decrease in the fluorescence intensity after irradiation with light that was indicative of occurrence of strand exchange reaction. On the other hand, the sample containing the photo-responsive nucleic acid showed a decrease in the fluorescence intensity to as low as about 97% by irradiation with light (see the bar labelled with “+azobenzene-modified DNA” in FIG. 14). The sample containing the photo-responsive nucleic acid and PLL-g-Dex showed a decrease in the fluorescence intensity to as low as about 90% by irradiation with light (see the bar labelled with “+azobenzene-modified DNA+PLL-g-Dex” in FIG. 14). Thus it was found that the photo-responsive nucleic acid containing dimethylazobenzene could control strand exchange reaction of the double stranded nucleic acid in a light irradiation-dependent manner. It was also found that the efficiency of strand exchange could be improved by addition of PLL-g-Dex. In the present Example, the efficiency of strand exchange was about 10% at maximum under the condition where the concentration of the strand (unmodified DNA) to be replaced in the double stranded nucleic acid is the same as the concentration of the single stranded replacing nucleic acid. The efficiency of strand exchange may be further improved by increasing the concentration of the single stranded replacing nucleic acid in the reaction system.

### Example 7

### Amplification Reaction of Double Stranded Nucleic Acid Using Photo-Responsive Nucleic Acid

In the present Example, whether or not a double stranded nucleic acid could be amplified by formation of a linked nucleic acid utilizing strand exchange reaction using a photo-responsive nucleic acid was evaluated. Specifically, a series of the following reactions is studied. First a single stranded nucleic acid serving as a template is associated with two polynucleotides so that the polynucleotides are adjacent. A double stranded nucleic acid is produced by ligating the polynucleotides to form a linked nucleic acid. The linked nucleic acid is dissociated from the template nucleic acid strand in the produced double stranded nucleic acid by utilizing strand exchange reaction using the photo-responsive nucleic acid to form the linked nucleic acid of two polynucleotides and the formation is repeated. In the present Example, two polynucleotides were ligated through chemical ligation utilizing chemical reaction between a phosphorothioate group and a iodothymidine group.

(1) Preparation of Measurement Samples

### (1-1) Photo-Responsive Nucleic Acid

In the present Example, the photo-responsive nucleic acid (dimethylazobenzene-modified DNA) as Example 1 was used.

### (1-2) Template Nucleic Acid

As a template nucleic acid, an unmodified single stranded DNA with the base sequence of 40 bases fully complementary to the base sequence of the photo-responsive nucleic acid was synthesized by Japan Bio Services Co., Ltd. The sequence of the single stranded DNA is shown below:

(SEQ ID NO: 11)
5′-TAAGTACACTAGATCACTCAGGTATATCTCCTTCTTAAAG-3′

### (1-3) Polynucleotides

As polynucleotides, a single stranded DNA (first polynucleotide) having 20 bases labelled with a phosphorothioate group at the 3′-terminal and a single stranded DNA (second polynucleotide) having 20 bases labelled with an iodothymidine group at the 5′-terminal and with TexasRed® at the 3′-terminal were used. The first polynucleotide and the second polynucleotide have the base sequence fully complementary to the base sequence of SEQ ID NO: 11 when the first polynucleotide and the second polynucleotide are linked. The polynucleotides were synthesized by Japan Bio Services, Co., Ltd. The sequences of the polynucleotides are shown below:

First polynucleotide:
(SEQ ID NO: 12)
5′-CTTTAAGAAGGAGATATACC
phosphorothioate group-3′
Second polynucleotide:
(SEQ ID NO: 13)
5′-iodothymidine group-
TGAGTGATCTAGTGTACTTA-TexasRed®-3′

In the present Example, when the first polynucleotide and the second polynucleotide associating with the template nucleic acid are linked to produce the linked nucleic acid, a nucleic acid strand having 40 bases modified with TexasRed® is produced. Thus in the present Example, whether the nucleic acid was amplified or not can be evaluated by measuring the fluorescence intensity of the nucleic acid strand having 40 bases.

### (1-4) Preparation of Measurement Samples

The photo-responsive nucleic acid (final concentration: 0.75 nM), the template nucleic acid (final concentration: 1 nM) and PLL-g-Dex (molecular weight of PLL: 8000, graft ratio: 90%, final concentration: 15.6 μM) were dissolved in 10 mM phosphate buffer (pH 7) containing sodium chloride (0.15 M) and dithiothreitol (1 mM). The obtained solution was distributed into a fused silica 8-microwell array (Shimadzu Corporation) which was then heated to 60° C. on a Thermo Plate (Tokai Hit Corporation). As a control, a solution without the photo-responsive nucleic acid was also prepared, distributed into the 8-microwell array and heated to 60° C. as well. The first polynucleotide and the second polynucleotide were added to the wells at a final concentration of 1 nM respectively to prepare measurement samples.

(2) Amplification Reaction

At 5 minutes after addition of the polynucleotides, the array was irradiated for 1 minute with light (wavelength: 330 to 385 nm) which was emitted from a light source of a mercury lamp (ultra-high pressure UV lamp USH-1030L, Olympus Corporation) and passed through a ultraviolet light filter (U-MWU2, Olympus Corporation). At 30 minutes after irradiation with ultraviolet light, the array was irradiated for 1 minute with light (wavelength: 470 to 495 nm) which was emitted from a light source of the mercury lamp and passed through a visible light filter (U-MNIBA3, Olympus Corporation). For comparison, a sample which underwent 5 cycles of the series of light irradiation and a sample without light irradiation were prepared.

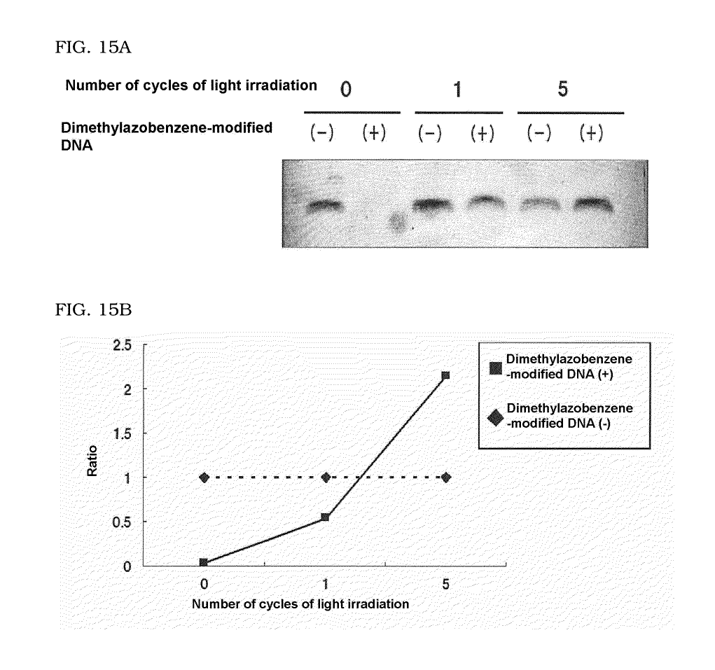

Samples (10 μL each) were collected from the wells, added with 1 M poly(vinyl sulphate) potassium salt solution at a final concentration of 0.1 M and left to stand at 4° C. for 90 minutes. The samples were further added with an equivalent volume of a formamide loading solution (95% formamide, NaOH, 2% bromophenol blue) and heated at 95° C. for 5 minutes to give samples for electrophoresis. The obtained samples were subjected to electrophoresis on a 20% acrylamide gel containing 4% urea (300 V, 30 minutes). A fluorescent image of the gel after electrophoresis was collected on a Molecular Imager (Bio-Rad). The resulting fluorescent images were converted to tif files and the intensity of fluorescent signals was converted to the numerical values with the Image J software. The relative values of the fluorescence intensity were calculated for the samples containing the photo-responsive nucleic acid for each number of cycles of light irradiation with the fluorescence intensity of the sample without photo-responsive nucleic acid being 1. The fluorescent image of the gel and the graph of the fluorescence intensity are shown in FIGS. 15A and 15B, respectively.

(3) Results

In the sample without the photo-responsive nucleic acid, the polynucleotides were associated with the template nucleic acid, so that the linked nucleic acid was formed independently of light irradiation. On the other hand, it was confirmed that in the sample containing the photo-responsive nucleic acid, the polynucleotides were not linked before irradiation with light while the polynucleotides were linked after irradiation with ultraviolet light and visible light for one time. Further, it was confirmed that four more cycles of irradiation with ultraviolet light and visible light increased the amount of the linked substance. Thus it is apparent that the double stranded nucleic acid can be amplified in a manner dependent to the number of light irradiation cycles by using the photo-responsive nucleic acid.

What is claimed is:

1. A method for controlling dissociation of a double stranded nucleic acid, comprising steps of:
irradiating a photo-responsive nucleic acid with light having a first wavelength to make the photo-responsive nucleic acid capable of associating with a first nucleic acid that is one of strands in the double stranded nucleic acid,
wherein the double stranded nucleic acid comprises the first nucleic acid and a second nucleic acid comprising a sequence complementary to the first nucleic acid, and
wherein the photo-responsive nucleic acid has a sequence complementary to the first nucleic acid; and
allowing complementary association of the photo-responsive nucleic acid with the first nucleic acid to dissociate the second nucleic acid from the first nucleic acid.

2. The method according to claim 1, further comprising steps of:
irradiating the photo-responsive nucleic acid with light having a second wavelength that is different from the first wavelength to make the photo-responsive nucleic acid incapable of associating with the first nucleic acid; and
dissociating the photo-responsive nucleic acid from the first nucleic acid.

3. The method according to claim 1, wherein the photo-responsive nucleic acid comprises one or more groups of at least one species selected from azobenzene and a derivative thereof.

4. The method according to claim 3, wherein the photo-responsive nucleic acid is made capable of associating with the first nucleic acid by transforming at least one species selected from azobenzene and a derivative thereof from a cis form to a trans form by irradiation with light having the first wavelength.

5. The method according to claim 3, further comprising steps of:
irradiating the photo-responsive nucleic acid with light having a second wavelength that is different from the first wavelength to make the photo-responsive nucleic acid incapable of associating with the first nucleic acid; and
dissociating the photo-responsive nucleic acid from the first nucleic acid,
wherein the photo-responsive nucleic acid is made incapable of associating with the first nucleic acid by transforming at least one species selected from azobenzene and a derivative thereof from a trans form to a cis form by irradiation with light having the second wavelength.

6. The method according to claim 3, wherein the derivative of azobenzene is dimethylazobenzene.

7. The method according to claim 1, wherein the steps are carried out in the presence of a strand-exchange-enhancing substance.

8. The method according to claim 7, wherein the strand-exchange-enhancing substance is at least one species selected from a cationic homopolymer and a cationic copolymer.

9. The method according to claim 8, wherein the cationic copolymer is a poly(L-lysine)-graft-dextran copolymer (PLL-g-Dex).

10. The method according to claim 7, wherein the photo-responsive nucleic acid is a nucleic acid comprising dimethylazobenzene attached thereto and the strand-exchange-enhancing substance is PLL-g-Dex.

11. The method according to claim 1, further comprising:
allowing complementary association of the second nucleic acid with a third nucleic acid.

12. A method for controlling strand exchange reaction of a double stranded nucleic acid, comprising steps of:
irradiating a photo-responsive nucleic acid with light having a first wavelength to make the photo-responsive nucleic acid capable of associating with a first nucleic acid that is one of strands in the double stranded nucleic acid,
wherein the double stranded nucleic acid comprises the first nucleic acid and a second nucleic acid comprising a sequence complementary to the first nucleic acid, and
wherein the photo-responsive nucleic acid comprises a sequence complementary to the first nucleic acid;
allowing complementary association of the photo-responsive nucleic acid with the first nucleic acid to dissociate the second nucleic acid from the first nucleic acid;
irradiating the photo-responsive nucleic acid with light having a second wavelength that is different from the first wavelength to make the photo-responsive nucleic acid incapable of associating with the first nucleic acid;
dissociating the photo-responsive nucleic acid from the first nucleic acid; and
allowing complementary association of the first nucleic acid with a third nucleic acid.

13. A method for amplifying nucleic acid by repeating following steps (1) to (4) under substantially isothermal condition, comprising steps of:
(1) allowing complementary association of a first polynucleotide with a single stranded target nucleic acid and allowing complementary association of a second polynucleotide with a region in the target nucleic acid that is adjacent to the region where the first polynucleotide associates;
(2) linking the first polynucleotide and the second polynucleotide;
(3) bringing a double stranded nucleic acid of the target nucleic acid and a linked strand of the first polynucleotide and the second polynucleotide into contact with a photo-responsive nucleic acid that has been made capable of associating with the target nucleic acid by irradiation with light having a first wavelength to allow complementary association of the target nucleic acid with the photo-responsive nucleic acid and to dissociate the linked strand; and
(4) irradiating the photo-responsive nucleic acid with light having a second wavelength that is different from the first wavelength to make the photo-responsive nucleic acid incapable of associating with the target nucleic acid and to dissociate the target nucleic acid from the photo-responsive nucleic acid,
wherein the dissociated target nucleic acid serves as the single stranded target nucleic acid in the step (1).

14. The method according to claim 13, wherein at least one of the first polynucleotide and the second polynucleotide comprises a label substance attached thereto,
wherein the method further comprises a step of detecting the label substance in the linked strand to validate amplification of the linked strand.

15. The method according to claim 13, wherein the photo-responsive nucleic acid comprises one or more groups of at least one species selected from azobenzene and a derivative thereof attached to the nucleic acid.

16. The method according to claim 15, wherein the photo-responsive nucleic acid is made capable of associating with the first nucleic acid by transforming at least one species selected from azobenzene and a derivative thereof from a cis form to a trans form by irradiation with light having the first wavelength.

17. The method according to claim 15, wherein the photo-responsive nucleic acid is made incapable of associating with the first nucleic acid by transforming at least one species selected from azobenzene and a derivative thereof from a trans form to a cis form by irradiation with light having the second wavelength.

18. The method according to claim 15, wherein the derivative of azobenzene is dimethylazobenzene.

19. The method according to claim 13, wherein the steps (1) to (4) are carried out in the presence of a strand-exchange-enhancing substance.

20. The method according to claim 19, wherein the strand-exchange-enhancing substance is at least one species selected from a cationic homopolymer and a cationic copolymer.


SEQUENCE LISTING (13 sequences)

SEQ ID NO: 1 - DNA, 40 bp, Artificial Sequence
  Synthetic photo-reactive oligonucleotide
ctttaagaaggagatatacctgagtgatctagtgtactta

SEQ ID NO: 2 - DNA, 40 bp, Artificial Sequence; identical to SEQ ID NO: 1
  Synthetic photo-reactive oligonucleotide

SEQ ID NO: 3 - DNA, 40 bp, Artificial Sequence; identical to SEQ ID NO: 1
  Synthetic control DNA

SEQ ID NO: 4 - DNA, 40 bp, Artificial Sequence; identical to SEQ ID NO: 1
  Synthetic oligonucleotide

SEQ ID NO: 5 - DNA, 41 bp, Artificial Sequence
  Synthetic oligonucleotide
ttaagtacactagatcactcaggtatatctccttcttaaag

SEQ ID NO: 6 - DNA, 40 bp, Artificial Sequence
  Synthetic control oligonucleotide
cagattacgattcaggtaaggcttagacttgaaaaccggt

SEQ ID NO: 7 - DNA, 40 bp, Artificial Sequence
  Synthetic control oligonucleotide
accggttttcaagtctaagccttacctgaatcgtaatctg

SEQ ID NO: 8 - DNA, 41 bp, Artificial Sequence; identical to SEQ ID NO: 5
  Synthetic oligonucleotide

SEQ ID NO: 9 - DNA, 20 bp, Artificial Sequence
  Synthetic oligonucleotide
ctttaagaaggagatatacc

SEQ ID NO: 10 - DNA, 20 bp, Artificial Sequence
  Synthetic oligonucleotide
tgagtgatctagtgtactta

SEQ ID NO: 11 - DNA, 40 bp, Artificial Sequence
  Synthetic template DNA
taagtacactagatcactcaggtatatctccttcttaaag

SEQ ID NO: 12 - DNA, 20 bp, Artificial Sequence
  Synthetic DNA probe
ctttaagaaggagatatacc

SEQ ID NO: 13 - DNA, 20 bp, Artificial Sequence
  Synthetic DNA probe
tgagtgatctagtgtactta